(19) United States
(12) Patent Application Publication
(10) Pub. No.: US 2024/0386198 A1
(43) Pub. Date: Nov. 21, 2024

(54) Method and System for Electronic Structural Medical Report Generation From Templates using Auto Impression that Interfaces with an Artificial Intelligence Interface (AI) to Analyze Images from Radiology Imaging Studies

(71) Applicant: Richard Scott Rosenblum, Phoenix, AZ (US)

(72) Inventor: Richard Scott Rosenblum, Phoenix, AZ (US)

(21) Appl. No.: 18/669,384

(22) Filed: May 20, 2024

Related U.S. Application Data
(60) Provisional application No. 63/467,672, filed on May 19, 2023.

(51) Int. Cl. G06F 40/186; G06F 3/0482; G06F 40/103; G06F 40/174; G06T 7/00; G16H 15/00

(52) U.S. Cl. CPC G06F 40/186; G06F 3/0482; G06F 40/103; G06F 40/174; G06T 7/0012; G16H 15/00; G06T 2207/10116

(57) ABSTRACT

Software system for electronic structural medical report generation from Templates using auto impression that interfaces with an artificial intelligence interface (AI) to analyze images from radiology imaging studies. Creating templates to generate reports more efficiently that are more accurate using pre-written/pre-structured phrases, sentences, and paragraphs. A gallery feature, where a user will open up the website, and go to the template the user has previously created. The user can pull up the “gallery” to quickly compare and contrast images all presumably from a reputable source, and that likely have caption available. If the user finds a photo that matches their current criteria, all they have to do is click on the photo or click on a button. If their current criteria match one of the photos in the gallery, the user can click on it, and it will populate the report with pre-written/pre-structured phrases that the user has previously entered.

### FEDERALLY SPONSORED RESEARCH

Not Applicable

### SEQUENCE LISTING OR PROGRAM

Not Applicable

### TECHNICAL FIELD OF THE INVENTION

The present invention relates generally to a software application for report generation. More specifically, the present invention relates to a software application for report generation in the medical field using Artificial Intelligence (AI) to analyze images from radiology imaging studies.

### BACKGROUND OF THE INVENTION

There is a need for many professions to create/generate written reports. Whether it is an attorney, doctor, engineer, psychologist, social worker, teacher, police, etc. Many of these reports have similar and somewhat repetitive language and format.

Recreating each report from scratch each time is inefficient use of time, energy, and money, and allows an unnecessary potential for errors.

In the past, users would either use a transcription service or type the report themselves, which was inefficient use of the users' time. Transcription required a relatively long delay where the transcriptionist would have to type a report out and return it to the user for editing. If it is a radiology report, referring doctors, especially emergency medicine doctors really appreciate a written report that is generated in real time, within a few minutes of the exam.

A more contemporary option is voice recognition. The biggest advantage to voice recognition is that the report is immediately available. Voice recognition companies advertise low error rates. However, the reality is that it is not error free and all it takes is one error of a key word and the entire report can be misconstrued or misinterpreted. This can also cause unnecessary legal liability. It can also create a lot of user stress and focus the user's attention away from the task at hand, and instead on proofreading their report. This, again, is an inefficient use of the user's time.

Templates can be created using pre-writing/pre-structured phrases, sentences, paragraphs, etc. They can be created using quick to use features like dropdown, pull down, radio, checkbox, etc. so the user can quickly, in a time efficient manner, create/generate a final report.

This can significantly reduce, and possibly eliminate errors (both voice recognition and typographical as well as contextual). Certainly, the error rate would be less than that of voice recognition software. The pre-written/pre-structured phrases can be well thought out and precisely worded so that it can be more precise and accurate. It can also significantly reduce the end users fatigue related to talking into a microphone for extended hours.

### DEFINITIONS

Unless stated to the contrary, for the purposes of the present disclosure, the following terms shall have the following definitions:

“Application software” is a set of one or more programs designed to conduct operations for a specific application. Application software cannot run on itself but is dependent on system software to execute. Examples of application software include MS WORD, MS EXCEL, a console game, a library management system, a spreadsheet system, a word processing system, etc. The term is used to distinguish such software from another type of computer program referred to as system software, which manages and integrates a computer's capabilities but does not directly perform tasks that benefit the user. The system software serves the application, which in turn serves the user.

The term “app” is a shortening of the term “application software”.

“Apps” are generally available through application distribution platforms, which began appearing in 2008 and are typically operated by the owner of the mobile operating system. Some apps are free, while others must be bought. Usually, they are downloaded from the platform to a target device, but sometimes they can be downloaded to laptops or desktop computers.

“API” In computer programming, an application programming interface (API) is a set of routines, protocols, and tools for building software applications. An API expresses a software component in terms of its operations, inputs, outputs, and underlying types. An API defines functionalities that are independent of their respective implementations, which allows definitions and implementations to vary without compromising each other.

The Domain Name System (DNS) is a hierarchical distributed naming system for computers, services, or any resource connected to the Internet or a private network. It associates various information with domain names assigned to each of the participating entities. Most prominently, it translates domain names, which can be easily memorized by humans, to the numerical IP addresses needed for the purpose of computer services and devices worldwide. The Domain Name System is an essential component of the functionality of most Internet services because it is the Internet's primary directory service.

“GUI”. In computing, a graphical user interface (GUI) sometimes pronounced “gooey” (or “gee-you-eye”)) is a type of interface that allows users to interact with electronic devices through graphical icons and visual indicators such as secondary notation, as opposed to text-based interfaces, typed command labels or text navigation. GUIs were introduced in reaction to the perceived steep learning curve of command-line interfaces (CLIs), which require commands to be typed on the keyboard.

The Hypertext Transfer Protocol (HTTP) is an application protocol for distributed, collaborative, hypermedia information systems. HTTP is the foundation of data communication for the World Wide Web. Hypertext is structured text that uses logical links (hyperlinks) between nodes containing text. HTTP is the protocol to exchange or transfer hypertext.

The Internet Protocol (IP) is the principal communications protocol in the Internet protocol suite for relaying datagrams across network boundaries. Its routing function enables internetworking, and essentially establishes the Internet.

An Internet Protocol address (IP address) is a numerical label assigned to each device (e.g., computer, printer) participating in a computer network that uses the Internet Protocol for communication. An IP address serves two principal functions: host or network interface identification and location addressing.

An Internet service provider (ISP) is an organization that provides services for accessing, using, or participating in the Internet.

A “mobile app” is a computer program designed to run on smartphones, tablet computers and other mobile devices, which the Applicant/Inventor refers to generically as “a computing device”, which is not intended to be all inclusive of all computers and mobile devices that are capable of executing software applications.

A “mobile device” is a generic term used to refer to a variety of devices that allow people to access data and information from wherever they are. This includes cell phones and other portable devices such as, but not limited to, PDAs, Pads, smartphones, and laptop computers.

A “module” in software is a part of a program. Programs are composed of one or more independently developed modules that are not combined until the program is linked. A single module can contain one or several routines or steps.

A “module” in hardware, is a self-contained component.

A “software application” is a program or group of programs designed for end users. Application software can be divided into two general classes: systems software and applications software. Systems software consists of low-level programs that interact with the computer at a considerably basic level. This includes operating systems, compilers, and utilities for managing computer resources. In contrast, applications software (also called end-user programs) includes database programs, word processors, and spreadsheets. Figuratively speaking, applications software sits on top of systems software because it is unable to run without the operating system and system utilities.

A “software module” is a file that contains instructions. “Module” implies a single executable file that is only a part of the application, such as a DLL. When referring to an entire program, the terms “application” and “software program” are typically used. A software module is defined as a series of process steps stored in an electronic memory of an electronic device and executed by the processor of an electronic device such as a computer, pad, smart phone, or other equivalent device known in the prior art.

A “software application module” is a program or group of programs designed for end users that contains one or more files that contain instructions to be executed by a computer or other equivalent device.

A “computer system” or “system” consists of hardware components that have been carefully chosen so that they work well together and software components or programs that run in the computer. The main software component is itself an operating system that manages and provides services to other programs that can be run in the computer. The complete computer is made up of the CPU, memory, and related electronics (main cabinet), all the peripheral devices connected to it and its operating system. Computer systems fall into two categories: clients and servers.

URL is an abbreviation of Uniform Resource Locator (URL), it is the global address of documents and other resources on the World Wide Web (also referred to as the “Internet”).

A “User” is any person registered to use the computer system executing the method of the present invention.

In computing, a “user agent” or “useragent” is software (a software agent) that is acting on behalf of a user.

A “web application” or “web app” is any application software that runs in a web browser and is created in a browser-supported programming language (such as the combination of JAVASCRIPT, HTML and CSS) and relies on a web browser to render the application.

A “website”, also written as Web site, web site, or simply site, is a collection of related web pages containing images, videos, or other digital assets. A website is hosted on at least one web server, accessible via a network such as the Internet or a private local area network through an Internet address known as a Uniform Resource Locator (URL). All publicly accessible websites collectively constitute the World Wide Web.

A “web page”, also written as webpage is a document, typically written in plain text interspersed with formatting instructions of Hypertext Markup Language (HTML, XHTML). A web page may incorporate elements from other websites with suitable markup anchors.

Web pages are accessed and transported with the Hypertext Transfer Protocol (HTTP), which may optionally employ encryption (HTTP Secure, HTTPS) to provide security and privacy for the user of the web page content. The user's application, often a web browser displayed on a computer, renders the page content according to its HTML markup instructions onto a display terminal. The pages of a website can usually be accessed from a simple Uniform Resource Locator (URL) called the homepage. The URLs of the pages organize them into a hierarchy, although hyperlinking between them conveys the reader's perceived site structure and guides the reader's navigation of the site.

### SUMMARY OF THE INVENTION

The present invention software delivered to a user as a website that is written to address the need for many professions to create/generate written reports. To allow the user to create templates that will allow the user to generate reports more efficiently that are more accurate using pre-written/pre-structured phrases, sentences, paragraphs, etc. for whatever profession, doctor, lawyers, teacher, psychologist, engineers, etc.

Furthermore, since its primary intent is for a doctor, such as a radiologist, the user can incorporate photos for any of the line items to provide visual comparisons to their current study they are interpreting and allow them to quickly enter the text if it is a match to their study.

In addition, there is a gallery feature, which is unique to this website. The intended idea is to benefit a Radiologist or Pathologist, but it can obviously be used in any profession requiring some sort of visual comparison or examples. In this scenario, the radiologist will open up the website, and go to the template the user has previously created for the temporal bone. The radiologist will pull up the “gallery” for the normal anatomy. The gallery will allow the radiologist to quickly compare and contrast images, all presumably from a reputable source, and that likely have captions available. If the radiologist finds a photo that matches their current study, all they have to do is click on the photo or click on a button.

Then the radiologist can review the gallery of fifty photos from radiology images of related pathology of this area the user previously uploaded. If their current exam matches one of the photos in the pathology gallery, they can just click on it, and it will populate the report with pre-written/pre-structured phrases that the user has previously entered.

In an alternative embodiment, the present invention teaches a method and system for electronic structural medical report generation from templates using auto impression that interfaces with an artificial intelligence interface (AI) such as but not limited to OPENAI to analyze images from radiology imaging studies.

In this embodiment, the Web App/software (WA) will use the previously described WA (Radiology templates) that involve allowing physicians to create radiology/medical reports using dropdown/pulldown and checkbox menus, populating reports with structured language.

The previous web app also described using image thumbnails to help the physician correctly identify and match imaging abnormalities and normal variate and will help the physician generate a radiology report that will more precisely and correctly make diagnosis, helping referring doctors chose best treatment options or surgeries, improving patient outcomes.

In this alternative embodiment, the webapp (WA) will interface with an Artificial Intelligence interface (AI) such as but not limited to OPENAI to analyze images from radiology imaging studies.

The AI will try to use published radiology criteria and images for defining normal anatomy or variations and abnormal pathology and differentiate them from each.

Once the AI has analyzed the images of the study it will display the images with the pathology outlined with a bright color, so the physician can identify it. The WA will generate a medical/radiology report from its analysis.

The radiologist will review the report and sign or edit the report for the final report. Once the report is signed the referring physician can go on the WA to review a video created by the AI showing the pathology, and they can review the video with the patient. The web app will allow the patient to sign a consent and acknowledgement that the patient reviewed it.

### BRIEF DESCRIPTION OF THE DRAWINGS

The accompanying drawings, which are incorporated herein form a part of the specification, illustrate the present invention and, together with the description, further serve to explain the principles of the invention and to enable a person skilled in the pertinent art to make and use the invention.

FIG. 1 is a flow chart illustrating the process of creating a template for one embodiment of the present invention.

FIG. 2 is the main menu GUI of one embodiment of the present invention.

FIG. 3 is the categories menu GUI of one embodiment of the present invention.

FIG. 4 is the sub-categories menu GUI of one embodiment of the present invention.

FIG. 5 is an example of the template titles under category XRAY and sub-category Musculoskeletal of one embodiment of the present invention.

FIG. 6 is an example of a user created report of one embodiment of the present invention.

FIG. 7 is an example of the drop down menu of one embodiment of the present invention.

FIG. 8 is an example of the pre-structured/planned words of one embodiment of the present invention.

FIG. 9 is an example of the shortcut creation of one embodiment of the present invention.

FIG. 10 is an example of the two selections the user has made to the right of the title of the features section of the GUI compared to FIG. 8 of one embodiment of the present invention.

FIG. 11 is an example of the final report output of one embodiment of the present invention.

FIG. 12 is a flow chart illustrating the process of creating a new or modified template for one embodiment of the present invention from an existing template.

FIG. 13 is an example of a user created template of one embodiment of the present invention.

FIG. 14 is a screenshot that demonstrates the auto impression and autonumbering features of the present invention.

FIG. 15 is a screenshot that demonstrates the Gallery feature of the present invention.

FIG. 16 is a flow chart of an alternative embodiment of the present invention that interfaces with an artificial intelligence interface (AI) to analyze images from radiology imaging studies.

### DETAILED DESCRIPTION OF THE INVENTION

In the following detailed description of the invention of exemplary embodiments of the invention, reference is made to the accompanying drawings (where like numbers represent like elements), which form a part hereof, and in which is shown by way of illustration specific exemplary embodiments in which the invention may be practiced. These embodiments are described in sufficient detail to enable those skilled in the art to practice the invention, but other embodiments may be utilized, and logical, mechanical, electrical, and other changes may be made without departing from the scope of the present invention. The following detailed description is, therefore, not to be taken in a limiting sense, and the scope of the present invention is defined only by the appended claims.

In the following description, numerous specific details are set forth to provide a thorough understanding of the invention. However, it is understood that the invention may be practiced without these specific details. In other instances, well-known structures and techniques known to one of ordinary skill in the art have not been shown in detail in order not to obscure the invention. Referring to the figures, it is possible to see the various major elements constituting the present invention.

The Webapp taught by the present invention is used by professionals like doctors and more specifically radiologists. The webapp also allows for users and administrators to create and edit templates for users to use.

There are user and admins that can created templates that correspond to studies done in the diagnostic imaging and other areas. The present invention allows the user to use a template with predefined semantics to generate reports, like radiology reports.

The present invention has preset and user defined semantics that make the doctor/radiologist much more accurate and specific with their language and therefore their diagnosis in their reports more succinct and more accurate.

There are a few main paragraph titles, including but not limited to CLINICAL INDICATION/HISTORY, TECHNIQUE, FINDINGS, and impression.

Each paragraph has the potential to have from one up to many vertical tabs that allow the user to select the appropriate option for that patient.

Each selection has a shortcut, short phrase the user will see in the webapp, an associated FINDINGS statement that goes in the FINDINGS paragraph, and associated IMPRESSION that is auto populated in the impression paragraph of the report.

The present invention teaches a novel feature called the thumbnail feature, which displays numerous thumbnail photos or diagrams that the user can select instead of the shortcut, to generate a report or part of a report.

The user can efficiently select which options apply to this particular patient's study they are reading and generate an accurate and succinct report.

The present invention uses novel features to make the template fast, easy, and efficient for the user to generate reports—i.e., drop-downs/pull-downs, radio buttons, checkboxes, etc. So, all the user has to do is click, click, click.

A gallery feature is a huge advantage, in some areas of art, specifically, radiology, imaging and pictures are crucial to performance of many jobs. To have pictures available so the radiologists have a so called “ruler” so the radiologist can see if an “inch” that he is diagnosing is same “inch” as a published article, is huge. The captions and text are prewritten into the template based on the caption and the article the pictures are from, so there is no inventing language at the view box.

When double clicked, it will copy the selected options in a formatted report, into the clipboard. This will eventually be pushed via direct communication to the PACS/RIS where the radiology reports are stored. Today this is called HL7 communication.

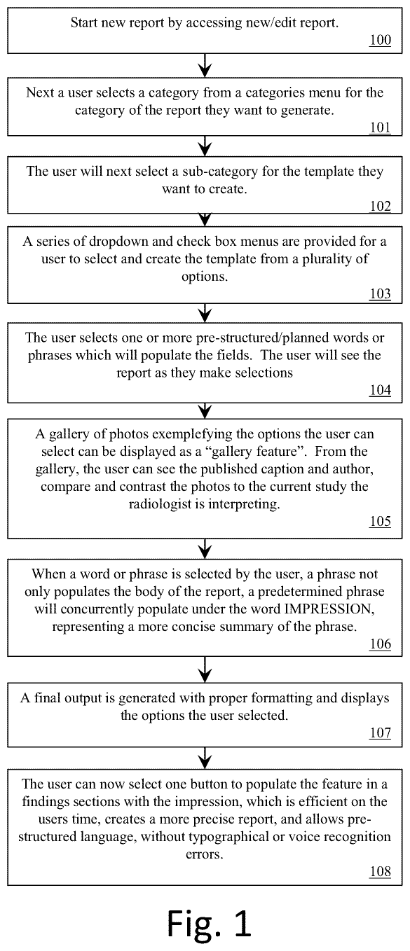

FIG. 1 is a flow chart illustrating the process of creating a template for one embodiment of the present invention. First a user starts a new report by accessing the new/edit report section of website 100. Next a user selects a category from a category's menu for the category of the report they want to generate 101. The user may also select a sub-category for the template they want to create 102. A series of drop down and check box menus are provided for a user to select and create the template from a plurality of options 103. The user selects one or more pre-structured/planned words which populates the fields and allows users to see the report as they make selections 104.

A gallery of photos exemplifying the options the user can select can be displayed as a “gallery feature”. From the gallery, the user can see the published caption and author, compare, and contrast the photos to the current study the radiologist is interpreting 105.

When a word or phrase is selected by the user, a phrase not only populates the body of the report, but a predetermined phrase will also concurrently populate under the word IMPRESSION, representing a more concise summary of the phrase. 106

Once a template is created, the user has the option to create a shortcut by placing a few brief words or criteria, which are unique to a dropdown selection. A final output is generated with proper formatting and displays the options the user selected 107. During the process of creating the template, the user has used an auto-impression feature through the pre-selection of language for particular options/fields. The user can now select one button to populate the feature in a finding's sections with the impression, which is efficient on the user's time, creates a more precise report, and allows pre-structured language, without typographical or voice recognition errors 108.

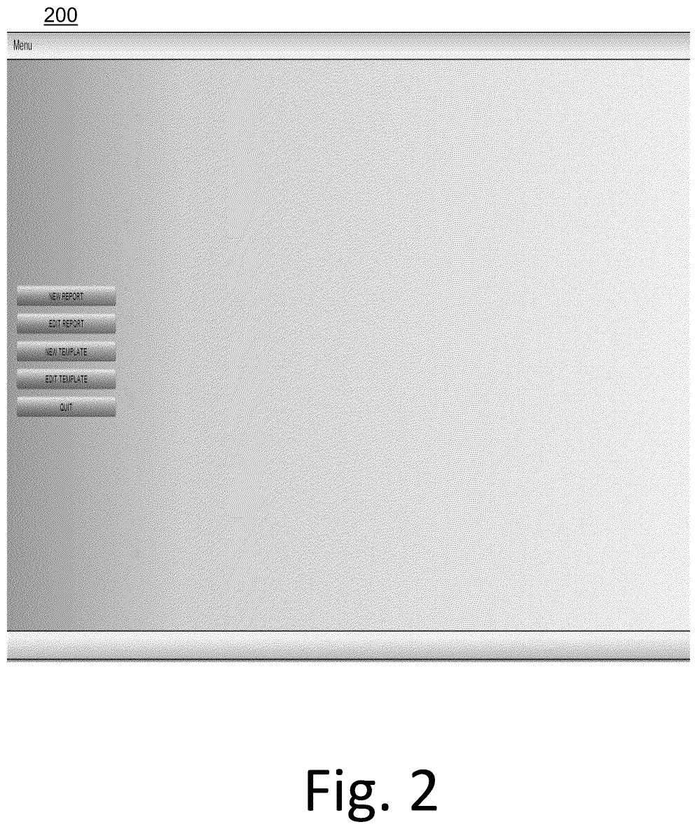

FIG. 2 is the main menu GUI 200 of one embodiment of the present invention. This is the main menu of the website for generating a new report. This will be the most common feature the user will use. Upon selecting this option, the user will be able to generate reports (in this exemplary embodiment, radiology reports), generally using a combination of dropdown and checkbox features with pre-structured language from previously created templates.

Radio buttons, drop down/pull down, check box, blanks, and constants are all features, most are commonly used in programming today. All of these features will be used to allow the user to quickly click to select pertinent pre-structured/pre-written phrases. Checkbox: This lets a user select from 0-infinite number of choices. C or C0 stands for 0-infinite. C1 stands for 1-infinite, where the user must select one feature. C1 (checkbox) was created to allow the user to place a negative default as the first line item. If the user selects no line items, then line item 1 will be used as default. If the user selects any other line item other than the first line item, the first one becomes unselected/unchecked.

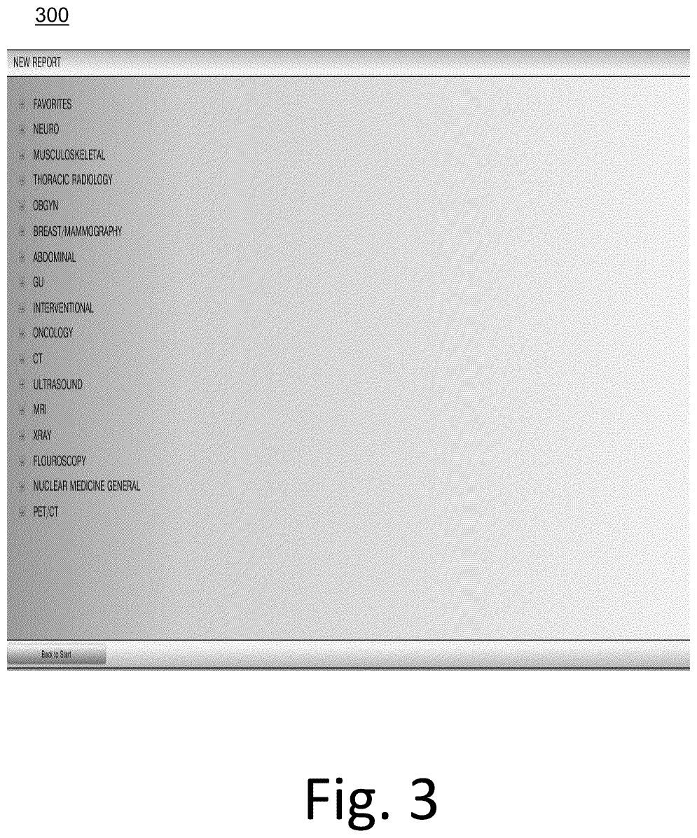

FIG. 3 is the categories menu GUI 300 of one embodiment of the present invention. In the radiology example and embodiment, this feature allows the user to see, and select a category of the report they want to generate.

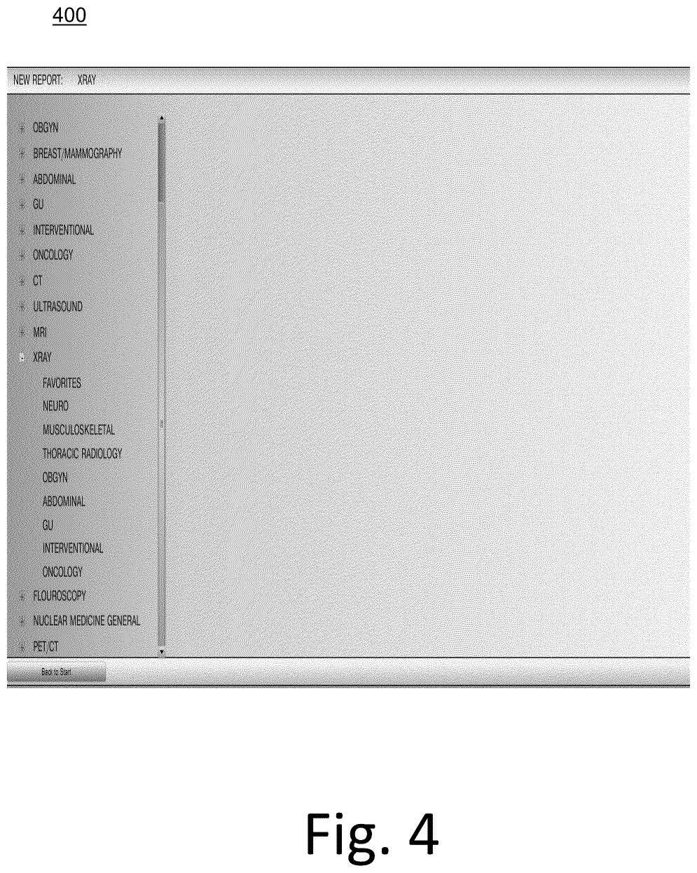

FIG. 4 is the sub-categories menu GUI 400 of one embodiment of the present invention. In the radiology exemplary embodiment, this feature allows the user to see, and select a subcategory of the report they want to generate. FIG. 4 is an example of the template titles under Category XRAY and subcategory Musculoskeletal. The user in this example selects the template named “XRAY KNEE NEW”.

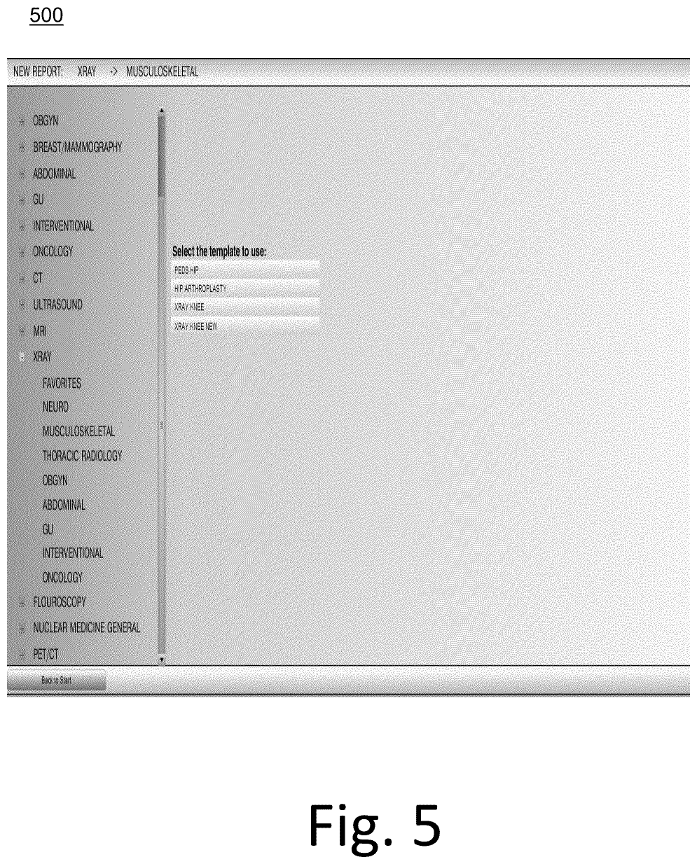

FIG. 5 is an example of the template titles under category XRAY and sub-category Musculoskeletal of one embodiment of the present invention. This is an example of a user created template 500. This one is called XRAY KNEE NEW.

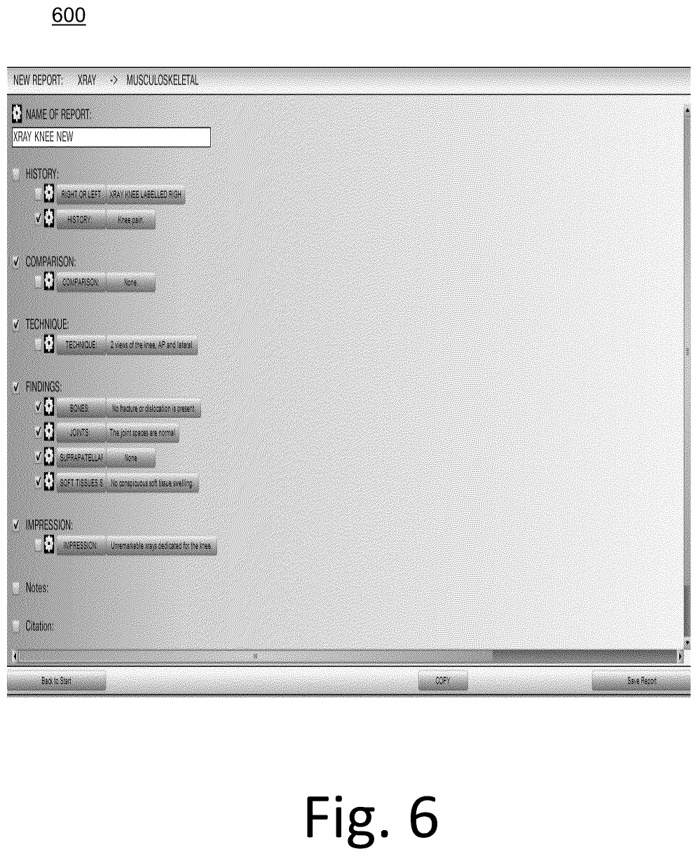

FIG. 6 is an example of a user created report 600 of one embodiment of the present invention. There are standard headers listed here that appear in most reports (HISTORY, TECHNIQUE, COMPARISON, FINDINGS, impression).

The checkbox to the left of the “Headers” and dropdown or checkbox feature titles is used to either allow or suppress these specific words to be included in the report.

The “gear” icon to the left is to edit things. This is more applicable in the EDIT TEMPLATE mode.

The title of each feature (dropdown or checkbox) is shown to the right of each “gear” icon within a button. To the right of that is a button containing the “default” selection for each feature (dropdown or checkbox).

Note, the impression header also displays the “default” impression.

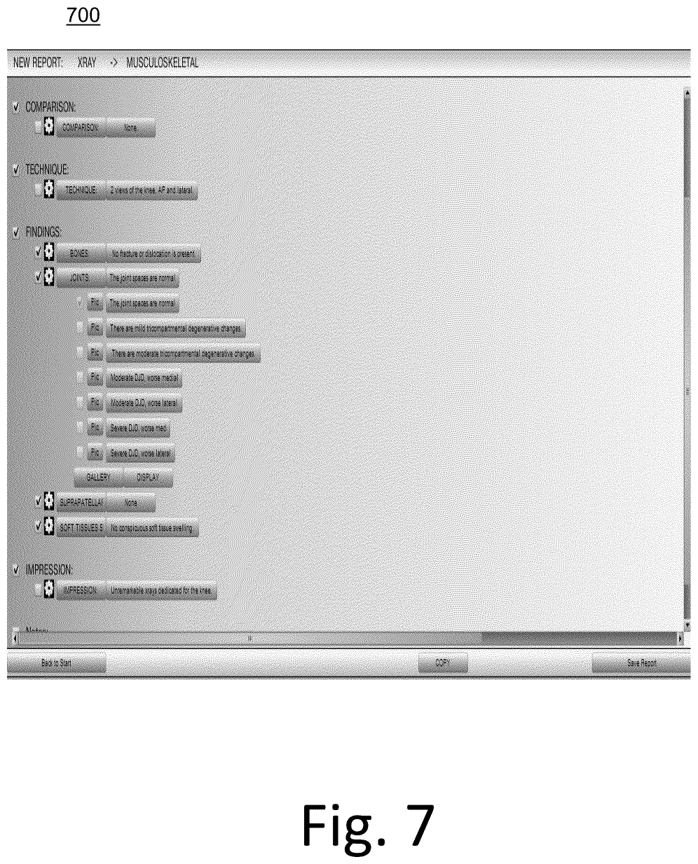

FIG. 7 is an example of the drop down menu of one embodiment of the present invention. In this screen capture 700, the user has opened up the “dropdown” called “JOINT” and is looking at his options. The user had previously used abbreviations called “shortcuts” to be displayed in the final four options. The intent of these is to minimize clutter in the NEW/EDIT REPORT modes or to allow the user to place criteria related to the text that will eventually populate the report.

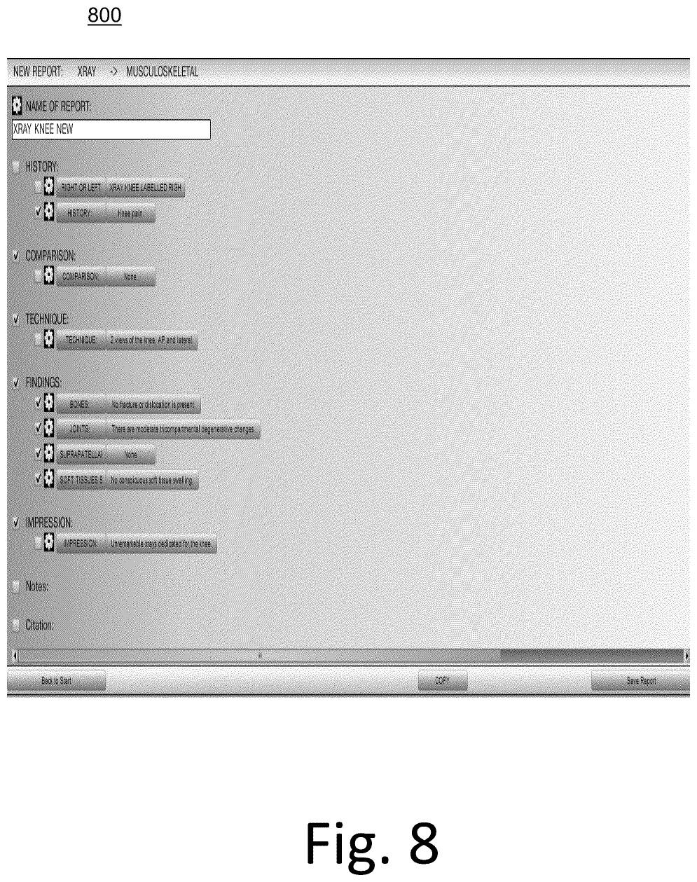

FIG. 8 is an example of the pre-structured/planned words of one embodiment of the present invention. In this screen 800 the user has selected the pre-structured/planned words “There are moderate tri-compartmental degenerative changes.” for this dropdown. It is populated to the right of the title of the feature so that the user can see the report as they make selections. This language will eventually populate the official radiology report.

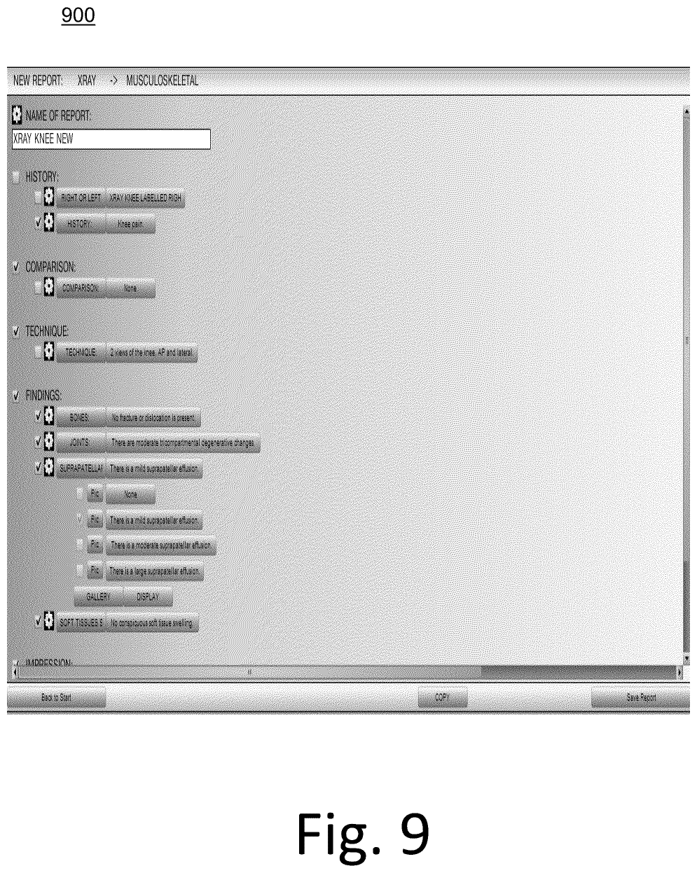

FIG. 9 is an example of the shortcut creation of one embodiment of the present invention. In this screenshot 900, the user has left clicked on the pulldown called “Suprapatellar effusion”. The pre entered options are displayed as a dropdown menu so the user can see what options of phrases are available. Sometimes the user will see here the actual text that will populate the body of the report. When creating templates, the user has an option to create a “shortcut”. In brief, the user can place a few key words, or criteria, which are unique to this selection in the dropdown.

The intent of this is to make the drop down/pull down, radio button, or checkbox feature look less cluttered. For example, let us say that the user has a radio button labelled car. The user wants output to be “The car has a green exterior”. For a shortcut for this line item, the user can just use the word “green”. So, in this specific down/pull down, radio button, or checkbox feature, the user will only see the word green. If this field is selected, the report/output will display “The car has a green exterior”.

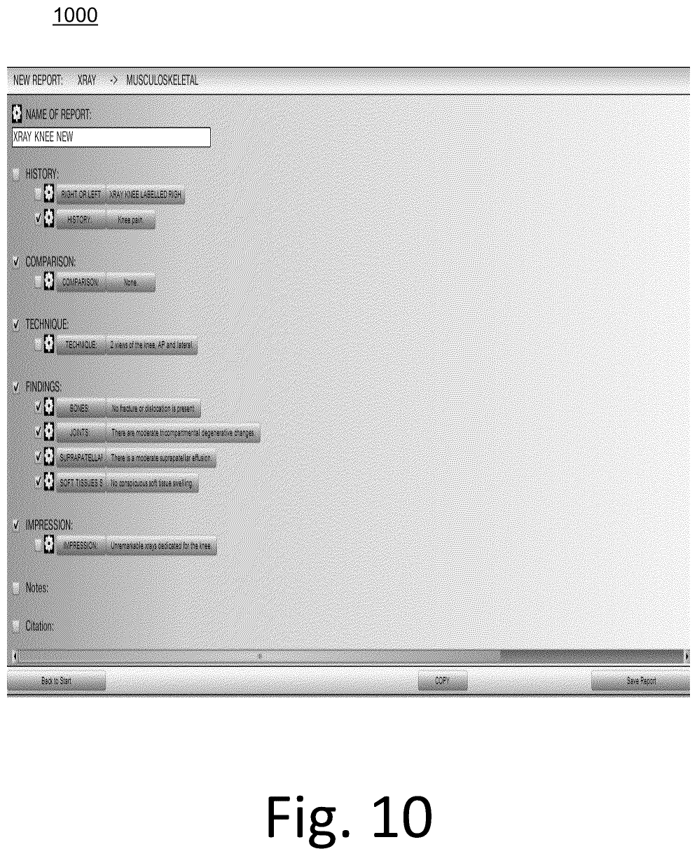

FIG. 10 is an example of the two selections the user has made to the right of the title of the features section of the GUI 1000 compared to FIG. 8 of one embodiment of the present invention. In this screenshot, you can see both selections the user had made to the right of the title of the features.

“There are moderate tri-compartmental degenerative changes” and “There is a moderate suprapatellar effusion”. These selections will eventually populate the final report.

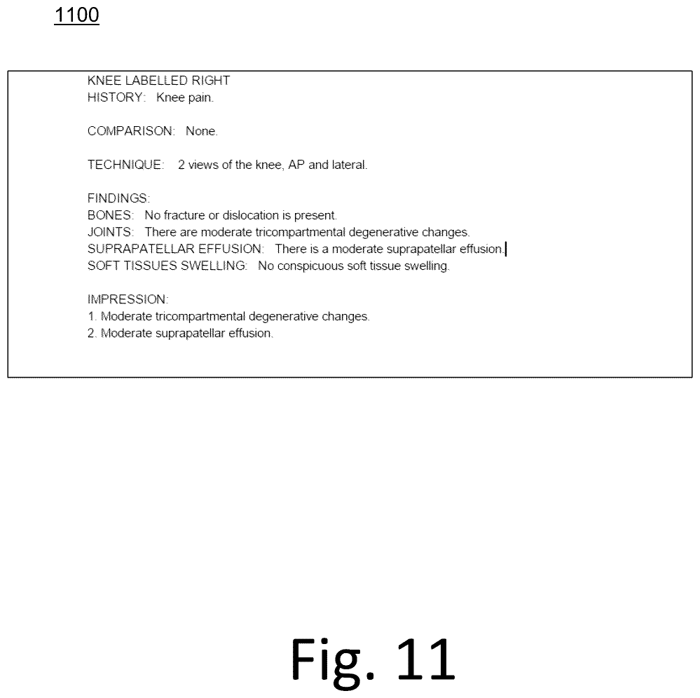

FIG. 11 is an example of the final report output 1100 of one embodiment of the present invention. This screenshot shows the final output, formatted, displaying the options the user selected. Note that these selections are placed to the right of the title of the feature. Note also, that a feature called “auto impression” has been used by the user. The user has preselected language in the process of creating this template to populate the impression, for that particular option/field is selected.

A user has the option of using an auto impression field. At the bottom of medical/radiology reports, it is normal to have an impression. This is like a conclusion. Normally, the doctor or radiologist rewords significant pertinent findings in the impression. Let us go back to the green car example above. The user can pre-structure/pre-write an impression such as “The exterior of the car here is green.” Whenever this line item is selected, “green”, these words, “The exterior of the car here is green.” will appear in the impression.

So, in essence, the user needs to click on one button to populate both the feature in the FINDINGS and in the impression, which is efficient on the user's time, creates a more precise report, and allows pre-structured language, without typographical or voice recognition errors.

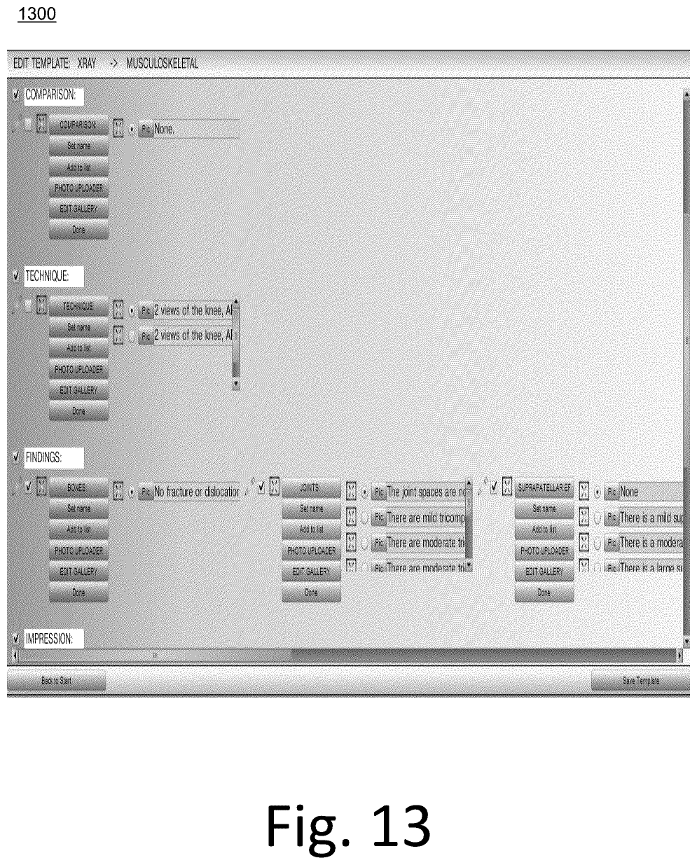

FIG. 13 is an example of a user creating template 1300 of one embodiment of the present invention. This is how the user structures a template.

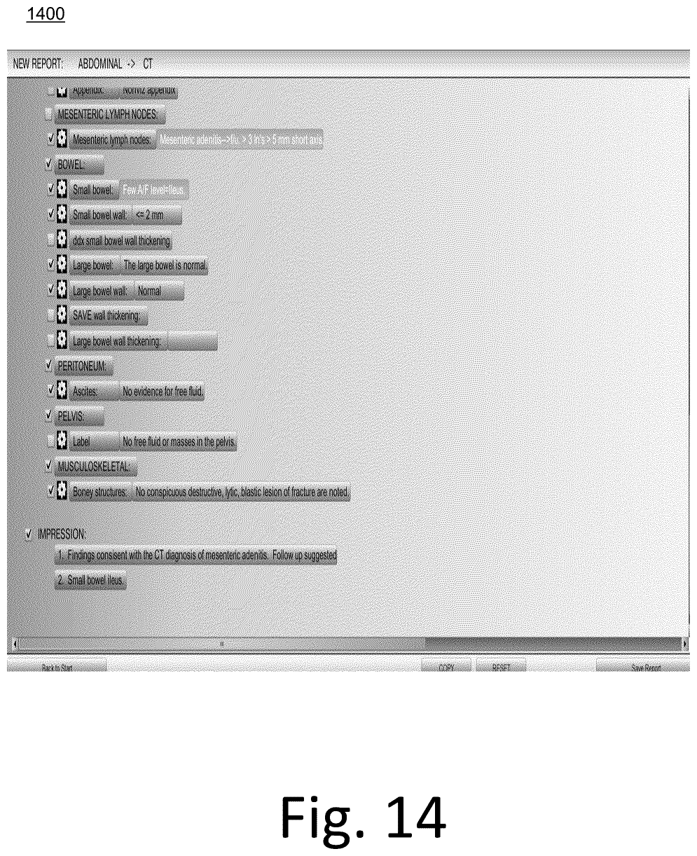

FIG. 14 illustrates a screen shot that demonstrates the AUTOIMPRESSION and AUTONUMBERING features 1400. When the user constructed this template, they filled text into a field called TEXT, which is the text that will populate the body of the report. They also filled the field called SHORTCUT, which is the text that, when present, will fill the button on the dropdown or checkbox feature. They also fill a field called auto impression. This is the text they would like to be populated as part of the IMPRESSION at the bottom of the report when they are generating a report from a template.

In the example above, the user selected an option referring to “mesenteric adenitis” and one referring to “ileus”. Upon clicking on these selection in the dropdown feature, they shortcut/button turns blue with white text, to assist the user to identify where he has made changes. The corresponding AUTOIMPRESSION field will populate below the word IMPRESSION AND the list will automatically be numbered with the AUTONUMBER feature, starting with 1, as is customary for a radiology report.

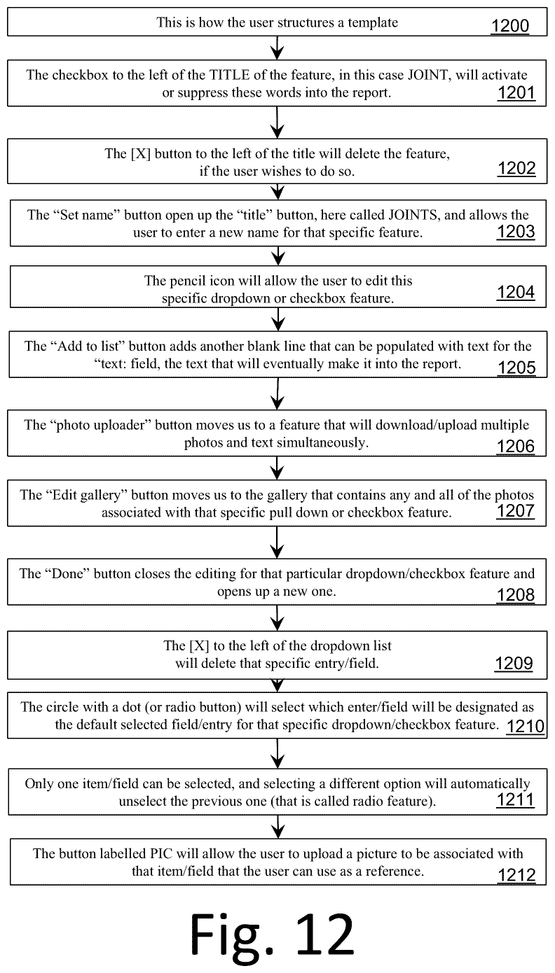

FIG. 12 is a flow chart illustrating the process of creating a new or modified template for one embodiment of the present invention from an existing template 1200. The “Joint” dropdown is now located to the right of the FINDINGS header. The checkbox to the left of the TITLE of the feature, in this case JOINT, will activate or suppress these words into report 1201. The [X] button to the left of the title will delete the feature if the user wishes to do so 1202. The “Set name” button opens up the “title” button, here called JOINTS, and allows the user to enter a new name for that specific feature 1203. The pencil icon will allow the user to edit this specific dropdown or checkbox feature 1204. The “Add to list” button adds another blank line that can be populated with text for the “text: field, the text that will eventually make it into the report 1205. The “photo uploader” button moves us to a feature that will download/upload multiple photos and text simultaneously 1206.

The “Edit gallery” button moves us to the gallery that contains any and all of the photos associated with that specific pull down or checkbox feature 1207, which is unique to the present invention. The intended idea is to benefit a Radiologist or Pathologist, but it can obviously be used in any profession requiring some sort of visual comparison or examples. This is easier to exemplify. In this example, a radiologist is interpreting a difficult study, a CT scan of the temporal bones. It is a difficult area to analyze because of the complex anatomy, and the fact that it is not commonly done. There are perhaps at least 30 structures that need to be identified, and about 50 different pathologies. All of these have very subtle differences that need to be identified in a small as 2 cm space.

The phrase “a picture is worth a thousand words” is an understatement faced with this challenge. In this scenario, the radiologist will open up the website, and go to the template the user has previously created for the temporal bone. The radiologist will pull up the “gallery” for the normal anatomy. The gallery will allow the radiologist to quickly compare and contrast images, all presumably from a reputable source, and that likely have captions available. If the radiologist finds a photo that matches their current study, all they have to do is click on the photo or click on the button (ENTER). Then the radiologist can review the gallery of 50 photos from pathology of this area the user previously uploaded. If their current exam matches one of the photos in the pathology gallery, they can just click on it, and it will populate the report with pre-written/pre-structured phrases that the user has previously entered.

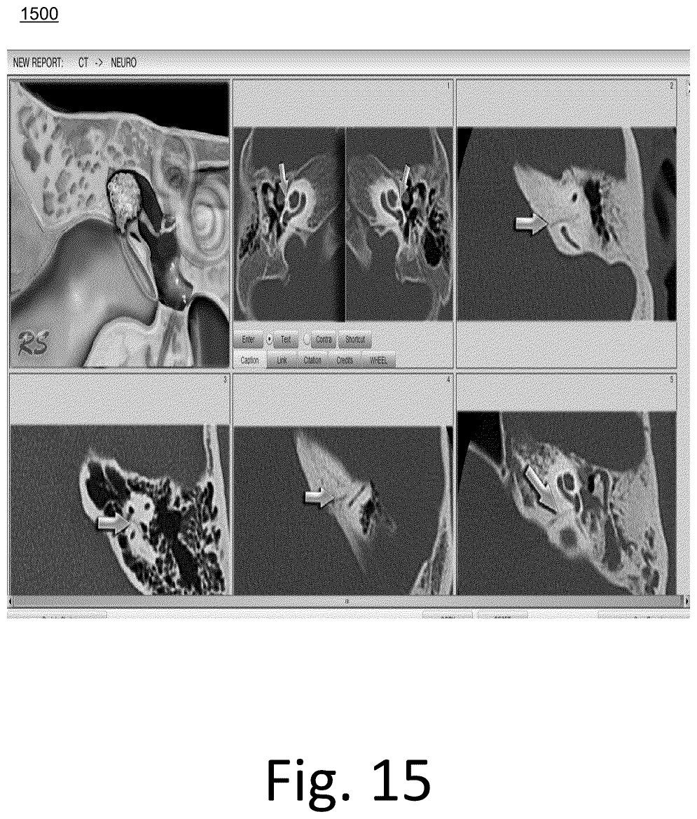

FIG. 15 shows an example of gallery feature 1500 with its functionality. These photos are part of a gallery, which can be attached to a single dropdown, checkbox, or radio button feature. Each photo is represented in that feature by one line of fields, SHORTCUT, TEXT, CONTRA, AUTOIMPRESSION, CREDITS, CITATION, AUTOIMPRESSION, LINK.

SHORTCUT—This button will allow the user to view/edit this field. Just as described earlier, this is the text that will populate the button within the dropdown/pulldown or checkbox feature. The user can select a few words or populate it with a criteria to represent the text in that line.

TEXT—This was previously described, and this button will allow the user to view/edit this field. This is the same text field described earlier, the text that will actually populate the body of the report generated.

CONTRA—This is the text field that the user has the option to use that describes the exact opposite from the text. For example, if the text states—“The car is green”, contra could say “The car is not green”.

LINK—This is the web address where the photo is located.

CITATION—this button will allow the user to view/edit the citation information to the article where this image is from.

CREDITS—This button will allow the user to view/edit this field. This field can have the authors names.

AUTOIMPRESSION—This was previously described, and this button will allow the user to view/edit this field.

ENTER (or double left click)—This button will populate the current report the user is creating with the TEXT FIELD corresponding to that particular photo/image.

Photos that the user uploads for his templates for a specific line within a DROPDOWN/PULLDOWN, RADIO BUTTON, or CHECKBOX feature will be visible here. The user can add fields that are relevant to the photo such as where it was obtained (Author, Article citation, link, caption, etc.). They can also add TEXT related to this photo. Upon selecting this photo (i.e., the user feels that this photo is relevant to the current active report they are generating, this specific pre-written/pre-structured text will enter into the position that will be used in the report. It is an action equal to selecting the actual text in that particular DROPDOWN/PULLDOWN, RADIO BUTTON, or CHECKBOX feature. The user can also magnify the image.

The “Done” button closes the editing for that particular dropdown/checkbox feature and opens up a new one 1208. The [X] to the left of the dropdown list will delete that specific entry/field 1209.

The circle with a dot (or radio button) will select which enter/field will be designated as the default selected field/entry for that specific dropdown/checkbox feature 1210. Only one item/field can be selected, and selecting a different option will automatically unselect the previous one (that is called radio feature) 1211. The button labelled PIC will allow the user to upload a picture to be associated with that item/field that the user can use as a reference 1212.

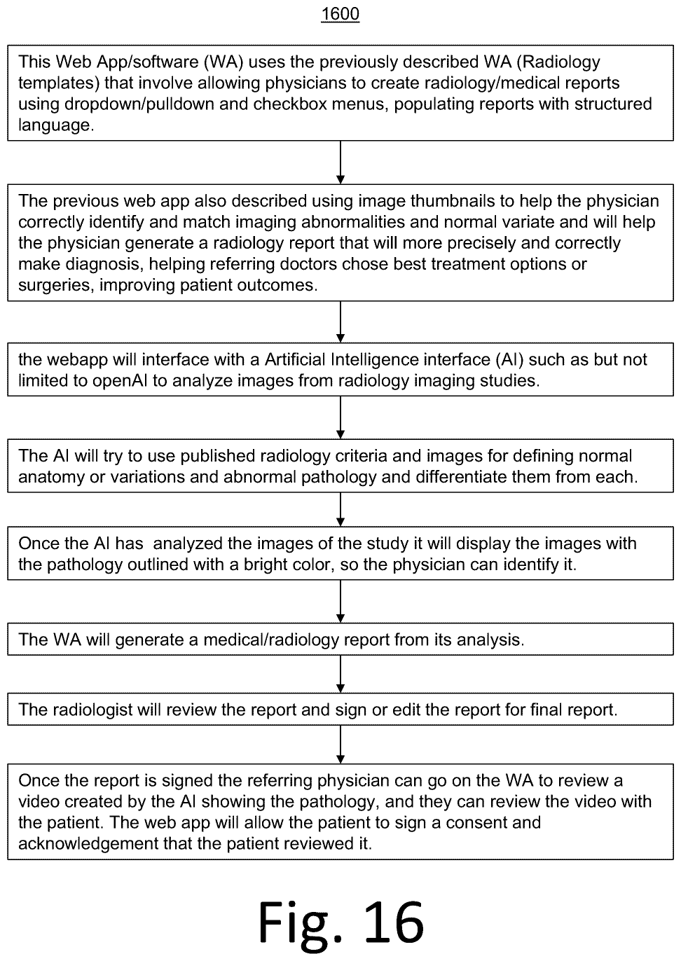

Now referring to FIG. 16, in an alternative embodiment, the present invention teaches a method and system for electronic structural medical report generation from templates using auto impression that interfaces with an artificial intelligence interface (AI) such as but not limited to OPENAI to analyze images from radiology imaging studies.

In this embodiment, the Web App/software (WA) will use the previously described WA (Radiology templates) that involve allowing physicians to create radiology/medical reports using dropdown/pulldown and checkbox menus, populating reports with structured language.

The previous WebApp, radiology templates were intended to make radiologists more accurate and precision and improving patient care not only throughout the USA but throughout the world, and reduce medical errors, and improve turnaround time for patients and referring doctors to receive their reports.

The previous web app also described using image thumbnails to help the physician correctly identify and match imaging abnormalities and normal variate and will help the physician generate a radiology report that will more precisely and correctly make diagnosis, helping referring doctors chose best treatment options or surgeries, improving patient outcomes.

In this alternative embodiment, the webapp (WA) will interface with an Artificial Intelligence interface (AI) such as but not limited to OPENAI to analyze images from radiology imaging studies.

The AI will try to use published radiology criteria and images for defining normal anatomy or variations and abnormal pathology and differentiate them from each.

In an alternative embodiment, the present invention the AI will specifically not try to identify calcifications on the aorta.

Once the AI has analyzed the images of the study it will display the images with the pathology outlined with a bright color, so the physician can identify it. The WA will generate a medical/radiology report from its analysis.

The radiologist will review the report and sign or edit the report for the final report. Once the report is signed the referring physician can go on the WA to review a video created by the AI showing the pathology, and they can review the video with the patient. The web app will allow the patient to sign a consent and acknowledgement that the patient reviewed it.

The system is set to run on a computing device or mobile electronic device. A computing device or mobile electronic device on which the present invention can run would be comprised of a CPU, Hard Disk Drive, Keyboard, Monitor, CPU Main Memory, and a portion of main memory where the system resides and executes. Any general-purpose computer, smartphone, or other mobile electronic device with an appropriate amount of storage space is suitable for this purpose. Computers and mobile electronic devices like these are well known in the art and are not pertinent to the invention. The system can also be written in a number of different languages and run on a number of different operating systems and platforms.

Although the present invention has been described in considerable detail with reference to certain preferred versions thereof, other versions are possible. Therefore, the point and scope of the appended claims should not be limited to the description of the preferred versions contained herein.

As to a further discussion of the manner of usage and operation of the present invention, the same should be apparent from the above description. Accordingly, no further discussion relating to the manner of usage and operation will be provided.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to. falling within the scope of the invention.

The embodiments of the invention in which an exclusive property or privilege is claimed are defined as follows:

1. A method for electronic structured medial report generation recorded on non-transitory computer-readable medium and capable of execution by a computer, the method comprising the steps of:
displaying a website or other electronic interface by a computer for electronic structured medial report generation;
starting a new report by accessing a new/edit report section of the website;
selecting a category from a categories menu for assigning a category to the new report;
selecting one or more pre-structured/planned words which populates a body of the new report fields;
displaying the new report as selections are made and added to the new report body;
creating a template;
offering one or more options for formatting and content for selection from a drop down menu; and
generating a final report output with pre-determined formatting and any selected options.

2. The method of claim 1, further comprising the steps of
displaying a series of drop down and check box menus; and
selecting a sub-category for the template.

3. The method of claim 1, further comprising the steps of
once the template is created, presenting the user the option to create a shortcut by selecting one or more words or criteria, which are unique to a dropdown selection from the dropdown menu.

4. The method of claim 1, further comprising the steps of
during the process of creating the template, the user has used an auto-impression feature defined as the pre-selection of language for particular options/fields; and
one button selection populates the auto-impression feature in a findings sections with the pre-selection of language for particular options/fields associated with the auto-impression feature, which generates pre-structured language in the report body.

5. The method of claim 1, further comprising the steps of
providing a template with predefined and preset and user defined semantics to generate reports;
creating and editing templates for users to use;
using a combination of dropdown, checkbox, and radio button features with pre-structured language from previously created templates; and
creating templates that correspond to studies done of diagnostic imaging and radiology reports.

6. The method of claim 1, wherein
feature paragraph titles include:
clinical indication/history,
technique,
findings, and
impression;
each paragraph has the potential to have from one or more vertical tabs that allow the user to select the appropriate option for a patient;
a checkbox associated with one or more headers or feature titles corresponding to the body contents of the report is generated and displayed;
the checkbox associated with each corresponding header or feature title is used to either allow or suppress these specific words to be included in the generated final report.

7. The method of claim 1, wherein
a title of each feature (dropdown or checkbox) is shown to the right of each “gear” icon within a button; and
to the right of the gear icon is a button containing the “default” selection for each feature (dropdown or checkbox).

8. The method of claim 1, wherein
the user had previously used abbreviations called “shortcuts” to be displayed in the final four options;
the user has selected the pre-structured/planned words for this dropdown;
it is populated to the right of the title of the feature so that the user can see the report as they make selections; and
this language will populate the official report.

9. The method of claim 1, wherein
one of the formatting and content options for selection from the drop down menu includes the selection of specific phrases; and
one or more pre entered options corresponding to phrases representing actual text that will populate the body of the report are displayed as a dropdown menu so the user can see what options of prepared phrases are available to populate the body of the report.

10. The method of claim 9, wherein
the user will see here the actual text that will populate the report.

11. The method of claim 10, wherein when creating templates
the user has an option to create a shortcut; and
a thumbnail feature, which displays numerous thumbnail photos or diagrams, that a user can select instead of the shortcut, to generate a report or part of a report.

12. The method of claim 1, wherein
the final output is formatted, displaying the options the user selected; and
these selections are placed to the right of the title of the feature.

13. The method of claim 1, further comprising
a feature called “auto-impression” that has been used by the user;
the user has preselected language in the process of creating this template to populate the impression, for that particular option/field is selected; and
a user has the option of using an auto-impression field.

14. The method of claim 1, further comprising the steps of
creating and saving one or more medical conclusions/impressions for later; and
selecting one or more medical conclusions/impressions for inclusion at the end of the report body.

15. A method and system for electronic structural medical report generation from templates using auto impression that interfaces with an artificial intelligence interface (AI) to analyze images from radiology imaging studies, recorded on non-transitory computer-readable medium and capable of execution by a computer, the method comprising the steps of:
providing a Web App/software displaying a website or other electronic interface by a computer for electronic structured medial report generation;
starting a new report by accessing a new/edit report section of the website;
selecting a category from a categories menu for assigning a category to the new report;
selecting one or more pre-structured/planned words which populates a body of the new report fields;
displaying the new report as selections are made and added to the new report body;
creating a template;
offering one or more options for formatting and content for selection from a drop down menu; and
generating a final report output with pre-determined formatting and any selected options;
displaying a series of drop down and check box menus;
selecting a sub-category for the template;
once the template is created, presenting the user the option to create a shortcut by selecting one or more words or criteria, which are unique to a dropdown selection from the dropdown menu;
during the process of creating the template, the user has used an auto-impression feature defined as the pre-selection of language for particular options/fields;
one button selection populates the auto-impression feature in a findings sections with the pre-selection of language for particular options/fields associated with the auto-impression feature, which generates pre-structured language in the report body; and
using a combination of dropdown, checkbox, and radio button features with pre-structured language from previously created templates.

16. The method of claim 15, further comprising
interfacing with an artificial intelligence interface to analyze images from radiology imaging studies.

17. The method of claim 16, wherein
the AI uses published radiology criteria and images for defining normal anatomy or variations and abnormal pathology and differentiate them from each.

18. The method of claim 17, wherein
once the AI has analyzed the images of the study, the AI interface will display the images with the pathology outlined with a bright color, so the physician can identify it.

19. The method of claim 18, wherein
the web app generates a medical/radiology report from its analysis.

20. The method of claim 19, wherein
a radiologist will review the report and sign or edit the report for the final report;
once the report is signed the referring physician can go on the WA to review a video created by the AI showing the pathology and can review the video with the patient; and
the web app allows the patient to sign a consent and acknowledgement that the patient reviewed it.
